(19) United States
(12) Patent Application Publication
(10) Pub. No.: US 2002/0111508 A1
(43) Pub. Date: Aug. 15, 2002

(54) Method for producing esters of citric acids

(76) Inventors: Klaus Bergrath, Oberhausen (DE); Thomas Klein, Oberhausen (DE); Hans Bohnen, Moers (DE)

Correspondence Address: BIERMAN MUSERLIAN AND LUCAS, 600 THIRD AVENUE, NEW YORK, NY, 10016

(21) Appl. No.: 10/066,499

(22) Filed: Jan. 31, 2002

(30) Foreign Application Priority Data
Feb. 13, 2001 (DE) 101 06 627.9

(51) Int. Cl. C07C 67/08; C07C 69/66

(52) U.S. Cl. 560/180

(57) ABSTRACT

A process for producing esters of citric acid from citric acid and linear or branched aliphatic monoalcohols of 4 to 14 carbon atoms wherein the essential characteristics are the use of a limited stoichiometric excess amount of alcohol relative to the acid employed and the immediate acylation of the unprocessed ester, i.e. the conversion of the unprocessed ester with an acylating agent for masking the OH— group without having previously subjected it to any purification operation.

### SUMMARY OF THE INVENTION

A method for producing esters of citric acid with linear or branched aliphatic-monoalcohols of 4 to 14 carbon atoms comprising inter alia by the use of a limited stoichiometric excess amount of alcohol relative to the acid employed distinguished by remarkable technical simplicity regarding the process. Moreover, it yields highly pure products which satisfy even the high quality demands such as are made, for example, when employing such esters as plasticizers for synthetic materials.

### STATE OF THE ART

Plasticizers are widely employed in synthetic materials, in coating means and sealing compositions as well as in vulcanized and unvulcanized items. Without reacting chemically, they enter into a physical interaction with highly polymerized thermoplastic substances, preferably through their dissolving and swelling capacity. Thereby, a homogeneous system is developed whose thermoplastic range is shifted toward low temperatures compared to the original polymers. As a consequence of the addition of plasticizers, inter alia, a material is obtained whose properties are optimized compared to the untreated starting material. For example, their capacity for changes of form, of elasticity and strength are increased and their hardness is decreased.

To open the maximum breadth of application for plasticizers, they must meet a number of generally valid criteria. In the ideal case, they should be odorless, colorless, be resistant to light and thermostable. There is, furthermore, the requirement that they are unaffected by water, fire-retarding, hardly combustible and have low volatility. In particular, plasticizers, intended for use in the area of food items and for applications in the field of medicine, must be completely harmless in terms of health. Lastly, the production of the plasticizers should be simple in view of the required technical equipment as well as also in view of the required method steps and, to satisfy environmental requirements, must avoid the formation of nonutilizable byproducts and harmful waste products.

On the basis of their excellent plasticizer properties, certain phthalates, thus esters of phthalic acid, are extensively used as additives for thermoplastic materials, in particular for polyvinyl chloride. However, the universal application of these esters is repeatedly and unfavorably opposed in the technical literature by health considerations. For example, their use is ruled out in connection with food, as packaging material and in other products, whose use is subject to special care for reasons of precautionary health measures. Among these are, for example, items of everyday use, such as housewares and objects used in providing and caring for children, including toys, as well as products used in the field of medicine. Therefore, as plasticizers for auxiliary and finished products comprised of thermoplastic materials, which are intended for these special fields of application, phthalates are not used but rather the esters of citric acid which are toxicologically harmless.

The production of citric acid esters conventionally takes place by reaction of the acid with an alcohol or, if the desired properties of the finished product require such, with a mixture of different alcohols. The conversion with an excess amount of alcohol (molar ratio of acid:alcohol >1:3.6) is carried out in the presence of a catalyst to ensure complete conversion of the acid within a reasonable reaction time. In addition to the use of an excess amount of alcohol, for attaining a high conversion (corresponding to a high product yield) can also serve the elimination of the water formed in the course of the reaction. Acids, such as sulfuric acid have been found to be useful as catalysts; the use of titanates for this purpose has also been described. The ester synthesis is followed by the removal of the catalyst and washing of the product with water. Thereupon, the excess amount of alcohol is separated from the reaction mixture and the free OH— group of the citric acid is subsequently acylated, for example with acetic acid or acetic acid anhydride, preferably again in the presence of a catalyst. In a subsequent reaction step, the acid anhydride or the acid originally employed or formed from the acid anhydride, is distilled off. This is succeeded by the neutralization of the catalyst, drying of the unprocessed citric acid ester and its purification by distillation.

The complex and expensive steps of neutralizing, washing and distilling are considered to be unavoidable for obtaining high-quality plasticizers based on citric acid esters. It should be taken into consideration that plasticizers must meet a number of criteria to open for them a maximum number of application fields. Thus, apart from the already discussed toxicologic properties, they should be resistant to low and high temperatures, unaffected by water, be hardly combustible and have low volatility. These properties are critically affected by the structure of the starting materials. Moreover, the plasticizers must meet high sensory requirements, i.e. it is demanded that they be odorless and colorless.

These properties are closely related to the purity of the synthesis products and impurities contained even in minute concentrations (i.e. in the ppm range) in the ester plasticizer can make its suitability for the intended application questionable. Although odor and color can be adapted to the desired requirements by adding additives, auxiliary substances are avoided since they can impair other properties of the plasticizers and/or restrict their feasibility of application, for example due to their incompatibility with the substrate. Lastly, the production of the plasticizers, should be simple, thus should comprise procedural steps which should be few in number, as simple as feasible and not demand costly equipment. Furthermore, it is expected that the production method also meets stringent environmental requirements and avoids the generation of waste materials, such as byproducts not further utilizable, and waste water which contains injurious substances.

### OBJECTS OF THE INVENTION

It is an object of the invention to provide an improved process for the production of citric acid esters with a reduced number of steps in high yields.

It is another object of the invention to provide improved citric acid esters of monoalcohols of 4 to 14 carbon atoms of high purity.

These and other objects and advantages will become obvious from the following detailed description.

### THE INVENTION

The process of the invention for producing esters of citric acid by reaction of citric acid with aliphatic alcohols in a molar excess in the presence of a catalyst. Wherein for each mole of citric acid used, up to 3.4 moles of a linear or branched monoalcohol of 4 to 14 carbon atoms in the molecule, is used with the esterification being carried out at temperatures of 110 to 140° C. in the presence of an acidic catalyst and an entrainment agent for removing the water formed in the course of the reaction as an azeotropic mixture. The reaction mixture is acylated immediately following at temperatures of up to 110° C., the excess amount of the acylating agent is distilled off as such or as a secondary product, then the acid catalyst is neutralized, the reaction mixture is washed with water and volatile fractions are removed from the mixture by distillation and the distillation residue is dried.

The method of operation is distinguished by high reliability not only in laboratory and test operations but also in industrial installations. It can be readily performed discontinuously as well as continuously and yields citric acid esters of high purity, which can be employed in various applications, particularly as plasticizers. For the last application, the excellent color properties, especially the remarkable color stability, should be emphasized.

A highly important feature of the method of the invention is the use of a molar excess of alcohol in the esterification reaction and especially the maintenance of a maximum value of 3.4 moles of alcohol for each mole of citric acid. Limiting the amount of alcohol permits acylating the hydroxy ester of citric acid immediately and to dispense with a number of process steps, such as neutralization, washing and distillation following the esterification, the maintenance of which according to the methods of prior art are indispensable for obtaining highly pure esters. Moreover, the limitation of the excess amount of alcohol avoids the occurrence of problems which were entailed in the operating steps of conversion and now are dispensable, for example, the annoying generation of foam during the washing and, succeeding it, in certain cases, for example in the production of tributylcitrate, inversion of the aqueous and organic phases, i.e. the separation of the aqueous phase above the organic one. Of special importance is also the reduction of thermal method steps. It is known that the thermal loading of the unprocessed ester product leads to cleavage products which lastingly contaminate the ester and call for demanding and expensive purification measures.

According to a preferred embodiment of the method of operation of the invention, for each mole of citric acid, 3.1 to 3.35 moles of monoalcohol are used. It has been found to be especially useful to use 3.2 to 3.3 moles of monoalcohol for each mole of citric acid. As the alcohol component for the production of esters in the process of the invention are suitable linear as well as also singly or multiply branched alcohols of any desired source of 4 to 14 carbon atoms in the molecule. Due to their ready availability, oxoalcohols, in particular, are used, i.e. alcohols which were produced by oxosynthesis, by conversion of monoolefins with carbon monoxide and hydrogen. As the esterification reactants, instead of uniform alcohols, alcohol mixtures can also be used, i.e. mixtures of structurally isomeric compounds or mixtures of alcohols of different molecular size, whereby it is possible to adjust the desired properties of the end product. In particular if they serve for producing ester plasticizers, the monoalcohols contain preferably 4 to 10 carbon atoms in the molecule, of which butanols, hexanols and octanols are of special importance.

As the temperature at which the conversion of citric acid and alcohol is being carried out, the range of 110 to 140° C. has been found to be useful. Lower teperatures are not excluded if, due to the particularity of the reactants or the reaction conditions, a sufficiently high reaction rate is attained or only partial reactions are intended. Higher temperatures are generally avoided to prevent the risk of decomposition of the starting substances and of byproducts and end products and thus the contamination of the ester, for example, by substances damaging to the color. The use of reduced or increased pressure during the reaction is possible, however, such an implementation of the method is limited to special cases.

To ensure economically justifiable reaction times, it is necessary to increase the rate of conversion of acid and alcohol by adding a catalyst. For this purpose, suitable conventional, catalytically effective substances, such as sulfuric acid, formic acid, polyphosphoric acid, methane sulfonic acid or p-toluene sulfonic acid are used which are dissolved in the reaction mixture or suspended as the pure compound or also in the form of a mixture of different substances. Preferred are sulfuric acid, methane sulfonic acid and p-toluene sulfonic acid, which in the used concentration are chemically indifferent to reactants and product, are available cost-effectively and can readily be removed from the reaction mixture. The amount of the catalyst used can vary over a wide range and it is possible to use, for example, 0.01 percent by weight up to 5 percent by weight of the catalyst relative to the reaction mixture. However, since amounts of catalysts hardly offer any advantages, the catalyst concentration is usually 0.01 to 1.0, preferably 0.01 to 0.5 percent by weight, each relative to the reaction mixture.

Removing the water of reaction from the reaction mixture, which, particularly in view of the limited excess amount of alcohol is required to shift the esterification equilibrium in favor of the ester, takes place with the aid of azeotrope formers (entrainment agents). For this purpose, conventionally organic solvents are selected, which form with water, corresponding mixtures having a boiling point in the range of the temperature of 110 to 140° C. Examples of suitable entrainers are hexane, cyclohexane, toluene and the isomeric xylenes and preferred is cyclohexane. The amount of entrainment agent required for the complete removal of the water can be determined simply from the water formation calculated according to the stoichiometry of the esterification reaction and from the composition of the binary azeotrope. It has been found useful to use the entrainment agent in excess, usefully in a fraction which is 50 to 200 percent by weight greater than the theoretically calculated quantity. By trapping and fractionating the mixture of entrainment agent and water which has been distilled off, the course of the reaction can be tracked in simple manner. The entrainment agent separated from the azeotrope can be returned directly to the reaction.

The triesters obtained by conversion of citric acid and alcohol still contain a free hydroxy group which must be masked to prevent the ester from undergoing undesirable reactions. It is customary to acylate this highly reactive functional group, i.e. to esterify it with an acid. Highly useful acylating agents have been found to be acetic acid anhydride and butyric acid anhydride, but the use of other acids or acid anhydrides is not excluded. The esterification of the OH— group takes place with acid or acid anhydride, which are used in excess amounts relative to the triester. It is advantageous to use for each mole of triester, 1.2 to 1.6 moles, preferably 1.3 to 1.5 moles, of a monocarboxylic acid (or the corresponding amount of an acid anhydride. It is useful to maintain reaction temperatures up to 110° C., with the particular current temperature to be selected depending on the reactivity of the ester and of the acylating agent. The temperature range of 60 to 80° C. is preferred. The reaction takes place in the presence of a catalyst. In general, it is not required to add specifically a catalyst to the reaction mixture since it still contains a sufficient amount of the catalyst from the formation of the triester, but, in special cases, the addition of a catalyst is not excluded.

Both reactions, esterification of the citric acid and acylation of the hydroxyl group, are carried out in the absence of a solvent. However, it is also possible to allow the reaction to proceed in the presence of a solvent if the individual conditions require or suggest such. Frequently, the entrainment agent also fulfills simultaneously the task of solvent.

According to the invention, only the reaction product resulting from the esterification and acylation is transferred for purification, in contrast to the method of operation of the prior art, which provides separate purification steps, each after the esterification and after the acylation. From the unprocessed esterification and acylation mixtures, first the excess amounts of the acylating agent, namely acid or acid anhydride, as well as esters formed by conversion of the acylating agent and the alcohol component of the esterification reaction, is distilled off. This separation of reactants and byproducts takes place in a conventional manner and special measures do not need to be taken. The distillation is followed by the neutralization of the unprocessed product which operating step serves for the removal of the catalyst used for the esterification and acylation. The neutralization takes place with the aid of alkaline reagents, such as sodium carbonate or sodium hydroxide, which are conventionally employed as aqueous solutions and are intimately mixed with the organic phase by stirring or any suitable manner. Subsequently, the aqueous and the organic phases are separated, the reaction mixture is washed with water and dried according to conventional methods to eliminate the last traces of moisture. For example, the water residues can be removed at moderately increased temperatures under decreased pressure or a stream of an inert gas such as nitrogen can be passed through the residue.

In the following examples, there are described several preferred embodiments to illustrate the invention. However, it should be understood that the invention is not intended to be limited to the specific embodiments.

### EXAMPLE

Production of Acetyl Tributyl Citrate

The esterification of the citric acid with butanol and the immediately following acylation of the triester with acetic acid anhydride is carried out in a heatable 2 liter flask equipped with stirrer, internal thermometer and a filling nozzle. Into the flask were placed 210.14 g (1 mole) of citric acid monohydrate, 244.57 g of (3.3 moles) of n-butanol and 1.0 g of methane sulfonic acid and 44.0 g of cyclohexane, and the mixture was stirred until a homogeneous solution obtained. The reaction mixture was then heated to 110° C. and maintained at this temperature for 13 hours while the reaction water was transferred out. 399.2 g of unprocessed ester were obtained which immediately, i.e. without prior preparation or purification, were mixed with 122.5 g of acetic acid anhydride (1.2 moles) and 1.0 g (0.01 mole) of methane sulfonic acid and heated for 2 hours at 60° C. The excess amount of acylating agent was distilled off at a temperature of approximately 60° C. and 1 hPa and the remaining ester was then neutralized. For the neutralization, to the acylated unprocessed ester, there were added 59.2 g of water and 80.2 g of an aqueous solution of 10 percent by weight NaOH. After separating the two phases, the organic fraction was washed with water in an amount of about 50% of its weight, thereupon freed of the main quantity of the water by phase separation and finally dried by heating for 2 hours to maximally 110° C. at a pressure of approximately 1 hPa to obtain 390.0 g of a colorless crystal-clear pure ester.

Various mofidications of the process of the invention may be made without departing from the spirit or scope thereof and it is to be understood that the invention is intended to be limited only as defined in the appended claims.

What is claimed is:

1. A process for the production of esters of citric acid comprising reacting citric acid with aliphatic alcohols of 4 to 14 carbon atoms in a molar excess in the presence of a catalyst, using for each mole of citric acid, up to 3.4 moles of the linear or branched monoalcohol or a mixture of such alcohols, the esterification reaction being carried out at a temperature of 110 to 140° C. in the presence of an acidic catalyst and an entrainment agent for removal of the water formed in the course of the reaction as an azeotropic mixture, the reaction mixture being immediately followed by acylation at a temperature up to 110° C., the excess amount of acylating agent being distilled off as such or as a secondary product, followed by the acidic catalyst being neutralized, the reaction mixture being washed with water, distilling off the volatile fractions from the mixture and the distillation residue is dried.

2. The process of claim 1, wherein for each mole of citric acid, 3.1 to 3.35, of monoalcohol are used.

3. The process of claim 2 wherein 3.2 to 3.3 moles of monoalcohol are used.

4. The process of claim 1 wherein monoalcohol has 4 to 10 carbon atoms.

5. The process of claim 4 wherein the selected monoalcohol is the group consisting of butanol, a mixture of isomeric butanols, hexanol, a mixture of isomeric hexanols, octanol and a mixture of isomeric octanols.

6. The process of claim 1 wherein the concentration of the catalyst in the esterification of the citric acid with the rronoalcohol is 0.01 to 1.0 percent by weight, relative to the reaction mixture.

7. The process of claim 6 wherein the concentration of the catalyst is 0.01 to 0.5 percent by weight.

8. The process of claim 1 wherein as the esterification catalyst is selected from the group consisting of sulfuric acid, methane sulfonic acid and p-toluene sulfonic acid.

9. The process of claim 1 wherein the entrainment agent from removing the water formed in the course of the esterification is selected from the group consisting of cyclohexane, toluene and isomeric xylenes.

10. The process of claim 1 wherein for the acylation for each mole of citric acid triester, 1.2 to 1.6 moles, of monocarboxylic acid or the anhydride of a monocarboxylic acid is used.

11. The process of claim 10 wherein 1.3 to 1.5 moles are used.

12. The process of claim 1 wherein the acylation is carried out at a temperature of 60 to 80° C.

13. The process of claim 1 wherein the acylating agent is acetic acid anhydride or butyric acid anhydride.
